(19) United States
(12) Patent Application Publication
(10) Pub. No.: US 2014/0018778 A1
(43) Pub. Date: Jan. 16, 2014

(54) EXTERNAL FIXATION SYSTEM FOR PERCUTANEOUS DRAINAGE PROCEDURES

(71) Applicant: Board of Regents of the University of Texas System, Austin, TX (US)

(72) Inventors: Jorge E. Lopera, San Antonio, TX (US); Justin Alexander Long, San Antonio, TX (US)

(73) Assignee: Board of Regents of the University of Texas System, Austin, TX (US)

(21) Appl. No.: 13/934,897

(22) Filed: Jul. 3, 2013

Related U.S. Application Data
(60) Provisional application No. 61/667,720, filed on Jul. 3, 2012.

(51) Int. Cl. A61M 27/00

(52) U.S. Cl. CPC A61M 27/00
USPC 604/540; 604/327

(57) ABSTRACT

An external fixation system for percutaneous drainage procedures may include a base. The base may include a first side. The first side may be positioned adjacent an exterior surface of a subject during use. The base may include a first coupler coupled to the first side of the base. The base may include a second side opposite the first side. The base may include an opening extending through the first coupler and the base such that the first coupler is in fluid communication with the second side of the base. The first coupler may be couplable to a conduit during use such that the conduit is in fluid communication with the second side of the base. In some embodiments, the second side of the base may include a second coupler configured to facilitate the attachment of a drainage bag or a mechanism to flush or evacuate a drainage catheter during use.

### PRIORITY CLAIM

This application claims priority to U.S. Provisional Application Ser. No. 61/667,720 entitled “EXTERNAL FIXATION SYSTEM FOR PERCUTANEOUS DRAINAGE PROCEDURES” filed Jul. 3, 2012, which is incorporated herein by reference in its entirety.

### BACKGROUND OF THE INVENTION

1. Field of the Invention

The invention generally relates to drainage tubes and specifically to methods of securing such tubes to patients.

2. Description of the Relevant Art

Drainage tubes (such as catheters) can be inserted into a body cavity, duct, or vessel, and are used to allow drainage and, in some instances allows the administration of fluids to the patient. In most uses, drainage tubes are thin, flexible tubes, though in some uses, they may be a larger, solid tube. A drainage tube is left inside the body, either temporarily or permanently, may be referred to as an indwelling drainage tube.

Many patients require long-term drainage procedures for diversion of the bile or urine. The current use of sutures and external fixation system to secure the drainage tube is not optimal due to the need for retention sutures with associated risks of pain and discomfort, frequent drainage tube dislodgement, skin infection and psychological reminder for the patient of the chronic disease by the presence of the external tube. It is desirable to have a convenient way of securing and replacing drainage tubes during long term treatments.

### SUMMARY

An external fixation system for percutaneous drainage procedures may include a base. The base may include a first side. The first side may be positioned adjacent an exterior surface of a subject during use. The base may include a first coupler coupled to the first side of the base. The base may include a second side opposite the first side. The base may include an opening extending through the first coupler and the base such that the first coupler is in fluid communication with the second side of the base. The first coupler may be couplable to a conduit during use such that the conduit is in fluid communication with the second side of the base. In some embodiments, the second side of the base may include a second coupler configured to facilitate the attachment of a drainage bag or a mechanism to flush or evacuate a drainage catheter during use.

### BRIEF DESCRIPTION OF THE DRAWINGS

Advantages of the present invention may become apparent to those skilled in the art with the benefit of the following detailed description of the preferred embodiments and upon reference to the accompanying drawings.

FIG. 1 depicts an embodiment of an external fixation system for percutaneous drainage procedures.

FIG. 2 depicts an embodiment of an external fixation system for percutaneous drainage procedures.

FIG. 3 depicts an embodiment of an external fixation system for percutaneous drainage procedures, wherein the external fixation system is coupled to a drainage catheter.

FIG. 4 depicts an embodiment of an external fixation system for percutaneous drainage procedures, wherein the external fixation system is coupled to a drainage catheter.

FIG. 5 depicts an embodiment of an external fixation system for percutaneous drainage procedures, wherein the external fixation system is coupled to a drainage catheter using sutures (the depiction shows the sutures before being tied off.

FIG. 6 depicts an embodiment of an external fixation system for percutaneous drainage procedures, wherein the external fixation system is coupled to a drainage catheter before the drainage catheter is positioned.

FIG. 7 depicts an embodiment of an external fixation system for percutaneous drainage procedures, wherein the external fixation system is coupled to a drainage catheter and “externally” to faux skin coupled to a drainage bag using a luer lock.

FIG. 8 depicts an embodiment of an external fixation system for percutaneous drainage procedures, wherein the external fixation system is coupled to faux skin using sutures.

FIG. 9 depicts an embodiment of an external fixation system for percutaneous drainage procedures and a drainage catheter, wherein the external fixation system is coupled to faux skin using sutures.

While the invention is susceptible to various modifications and alternative forms, specific embodiments thereof are shown by way of example in the drawings and may herein be described in detail. The drawings may not be to scale. It should be understood, however, that the drawings and detailed description thereto are not intended to limit the invention to the particular form disclosed, but on the contrary, the intention is to cover all modifications, equivalents and alternatives falling within the spirit and scope of the present invention as defined by the appended claims.

The headings used herein are for organizational purposes only and are not meant to be used to limit the scope of the description. As used throughout this application, the word “may” is used in a permissive sense (i.e., meaning having the potential to), rather than the mandatory sense (i.e., meaning must). The words “include,” “including,” and “includes” indicate open-ended relationships and therefore mean including, but not limited to. Similarly, the words “have,” “having,” and “has” also indicated open-ended relationships, and thus mean having, but not limited to. The terms “first,” “second,” “third,” and so forth as used herein are used as labels for nouns that they precede, and do not imply any type of ordering (e.g., spatial, temporal, logical, etc.) unless such an ordering is otherwise explicitly indicated. For example, a “third die electrically connected to the module substrate” does not preclude scenarios in which a “fourth die electrically connected to the module substrate” is connected prior to the third die, unless otherwise specified. Similarly, a “second” feature does not require that a “first” feature be implemented prior to the “second” feature, unless otherwise specified.

Various components may be described as “configured to” perform a task or tasks. In such contexts, “configured to” is a broad recitation generally meaning “having structure that” performs the task or tasks during operation. As such, the component can be configured to perform the task even when the component is not currently performing that task (e.g., a set of electrical conductors may be configured to electrically connect a module to another module, even when the two modules are not connected). In some contexts, “configured to” may be a broad recitation of structure generally meaning “having circuitry that” performs the task or tasks during operation. As such, the component can be configured to perform the task even when the component is not currently on. In general, the circuitry that forms the structure corresponding to “configured to” may include hardware circuits.

Various components may be described as performing a task or tasks, for convenience in the description. Such descriptions should be interpreted as including the phrase “configured to.” Reciting a component that is configured to perform one or more tasks is expressly intended not to invoke 35 U.S.C. §112, paragraph six, interpretation for that component.

The scope of the present disclosure includes any feature or combination of features disclosed herein (either explicitly or implicitly), or any generalization thereof, whether or not it mitigates any or all of the problems addressed herein. Accordingly, new claims may be formulated during prosecution of this application (or an application claiming priority thereto) to any such combination of features. In particular, with reference to the appended claims, features from dependent claims may be combined with those of the independent claims and features from respective independent claims may be combined in any appropriate manner and not merely in the specific combinations enumerated in the appended claims.

It is to be understood the present invention is not limited to particular devices or biological systems, which may, of course, vary. It is also to be understood that the terminology used herein is for the purpose of describing particular embodiments only, and is not intended to be limiting. As used in this specification and the appended claims, the singular forms “a”, “an”, and “the” include singular and plural referents unless the content clearly dictates otherwise. Thus, for example, reference to “a linker” includes one or more linkers.

### DETAILED DESCRIPTION

### Definitions

Unless defined otherwise, all technical and scientific terms used herein have the same meaning as commonly understood by one of ordinary skill in the art.

The term “connected” as used herein generally refers to pieces which may be joined or linked together.

The term “coupled” as used herein generally refers to pieces which may be used operatively with each other, or joined or linked together, with or without one or more intervening members.

The term “directly” as used herein generally refers to one structure in physical contact with another structure, or, when used in reference to a procedure, means that one process effects another process or structure without the involvement of an intermediate step or component.

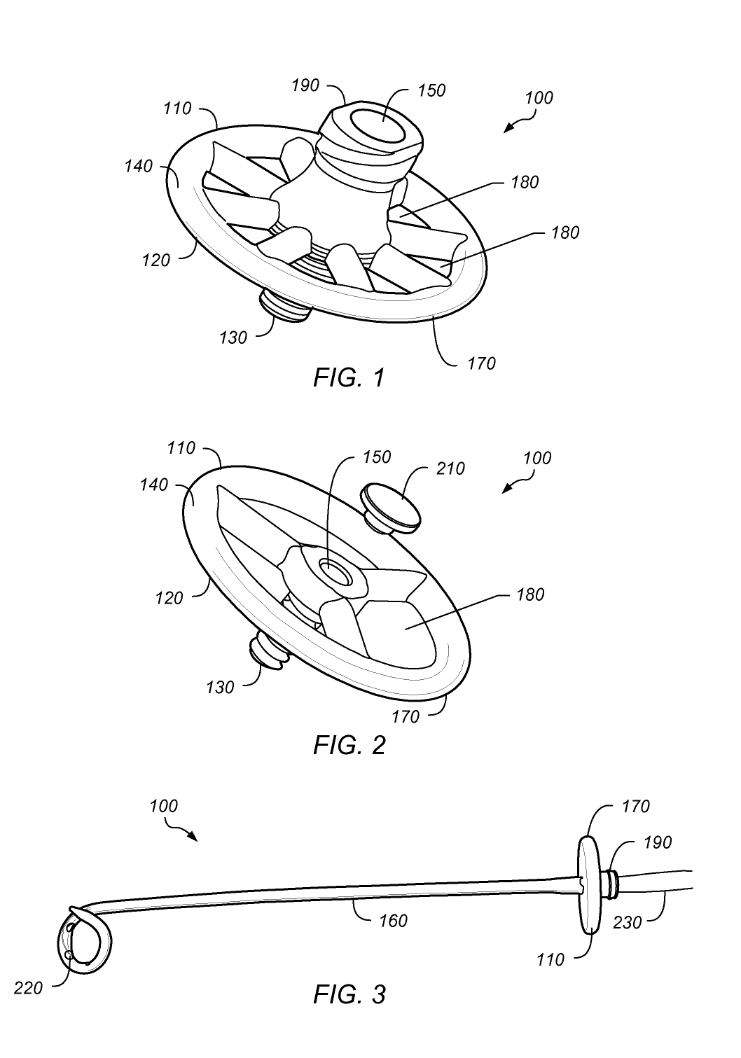

FIG. 1 depicts an embodiment of external fixation system 100 for percutaneous drainage procedures. External fixation system 100 for percutaneous drainage procedures may include base 110. The base may include first side 120. The first side may be positioned adjacent an exterior surface of a subject during use. The base may include first coupler 130 coupled to first side 120 of base 110. The base may include second side 140 opposite the first side. The base may include opening 150 extending through the first coupler and the base such that the first coupler is in fluid communication with the second side of the base. The first coupler may be couplable to conduit 160 during use such that the conduit is in fluid communication with the second side of the base.

In some embodiments, portions of the external fixation system may be formed from metallic and/or non-metallic materials. An external fixation system may be made of various materials including, but are not limited to, metals, metal alloys, silicon, plastic, polymers, ceramics and combinations thereof. Some external fixation system embodiments may include components made of materials that can be autoclaved and/or chemically sterilized. Some components of an external fixation system may be formed of materials unable to be autoclaved and/or chemically sterilized. Components unable to be autoclaved and/or chemically sterilized may be made of sterile materials and placed in working relation to other sterile components during assembly of an external fixation system. In some embodiments, an entire external fixation system may be made of materials that can be autoclaved and/or chemically sterilized so that the external fixation system is a reusable instrument. In other external fixation system embodiments, all or selected components of the external fixation system may be made of sterile, disposable materials so that all or components of the external fixation system are designed for single use.

In some embodiments, the base includes rim 170 extending out and around at least a portion of the base. The rim may function as a type of flange. The rim may function to inhibit the base from entering an interior of the subject as the rim will be larger than the opening through the skin of the subject. The rim may be in any number of shapes. The shape depicted in the accompanying drawings of a rim is that of a substantially circular shape but this should not be seen as limiting.

In some embodiments, the rim may include one or more openings 180 extending through the rim. The rim may include one or more openings 180 facilitating coupling of the base to the exterior surface of the subject during use. The openings may allow fasteners to be used to couple the rim and thereby the base to a subject. For example, sutures may be looped through the opening(s) coupling the rim to a subject. Other fasteners may be used, such as staples. In some embodiments, the base may be coupled to a subject using a hydrocolloid layer configured to absorb excess moisture around a perimeter of the base. In some embodiments, the base may be coupled to a subject using a double-sided hydrophilic adhesive layer configured to absorb excess moisture around the perimeter of the base. In some embodiments, the base may be coupled to a subject using a double-sided hydrophobic adhesive layer configured to repel excess moisture around the perimeter of the base.

The rim may include one or more openings such that cleaning of the exterior surface of the subject is facilitated during use. The openings may allow cleaning of an entry site at the skin level. The openings may allow a practitioner easier access to the skin below the rim allowing the practitioner to more easily disinfect the tissue around the base and rim.

In some embodiments, the base may include a membrane or luer lock connection in the back of the base to allow flushing of the tubes and/or connecting to an external drainage bag if needed. The base may include a fixation screw or lock in system to attach the base to the existing drain.

In some embodiments, the base may include a first coupler coupled to the first side of the base. In some embodiments, the first coupler may based upon a coupling mechanism including screw threads or coupling projections (e.g., teeth). Different attachments will allow the system to work with drains from 8-16 Fr. Once a conduit (e.g., a drainage catheter) is placed in the subject, an external portion is cut with sterile scissors. The cut end of the conduit may then be coupled to the base using the first coupler (e.g., a screw or a push in system). The base may be low profile and around 2 cm in diameter. The membrane in the center of the screw or fixation system will allow the patient to access the drain in case of blockage for flushing. In some embodiments, the base may be decoupled from the conduit. To exchange the conduits, the base is detached by, for example, either unscrewing or cutting the conduit adjacent to the base.

In some embodiments, the second side of the base may include second coupler 190 configured to facilitate the attachment of drainage bag 200 or a mechanism to flush or evacuate a drainage catheter during use. In FIG. 1 the second coupler includes a luer lock type of connection in the back to allow flushings or connection to a drainage bag. In some embodiments, the second coupler may include a coupling mechanism including external screw threads, internal screw threads, a female luer fitting, a male luer fitting, a push-to-connect fitting, or a spring-tension loaded trap-door fitting.

In some embodiments, base 110 may include a low profile design wherein, for example, the opening extending through the base may function as a second coupler. The opening may allow the connection of the base to a drainage bag and facilitates the ability to flush the drain using normal saline and a syringe without the luer lock design. In some embodiments, a closure mechanism may be used to inhibit conveyance of fluids through the opening when desired (for example, when there is no drainage bag hooked up to the opening). FIG. 2 depicts an embodiment of an external fixation system for percutaneous drainage procedures including closure mechanism 210. A closure mechanism may include detachable soft plug to keep the profile of the system more flat (e.g., as depicted in FIG. 2).

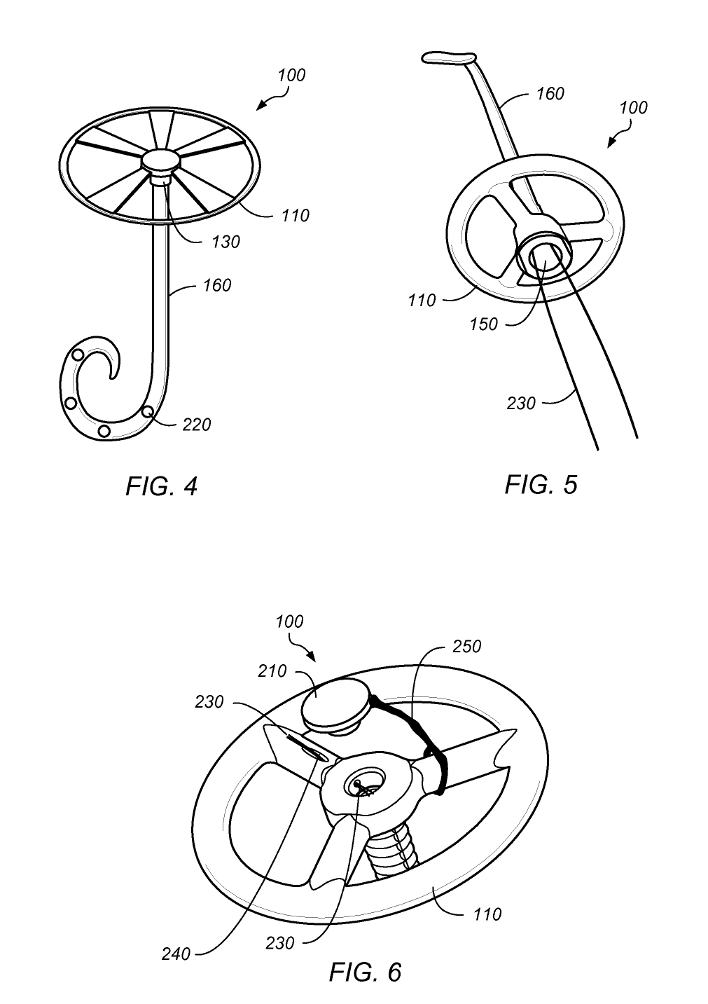

FIGS. 3-4 depict an embodiment of external fixation system 100 for percutaneous drainage procedures, wherein the external fixation system is coupled to conduit 160, specifically as depicted in these embodiments the conduit includes a drainage catheter. Drainage catheters may function to drain excess and/or unwanted fluids out of a subject. Drainage catheters may allow care givers a means to evaluate a subject's health at least as relates to the drainage site and the fluids drained. The drainage catheter may be at least semi-flexible so as not to harm a subject upon insertion. The drainage catheter may include a plurality of openings 220 such that during use fluids may enter the catheter through the openings such that the fluids may be conveyed out of the subject through the catheter.

As mentioned previously an external fixation system may be coupled to a conduit positionable internally in a subject such as a drainage catheter using a first coupler. In some embodiments, a conduit may be coupled to the base of the external fixation system using sutures. FIG. 5 depicts an embodiment of external fixation system 100 for percutaneous drainage procedures, wherein the external fixation system is coupled to a drainage catheter 160 using sutures 230 (the depiction shows the sutures before being tied off).

FIG. 5 depicts sutures 230 that lock the tip of the catheter exiting through the opening in the base externally relative to the subject. The sutures are then tied over a bar in the inside of the base and cut. This system will prevent the adjustment of the shape of the pigtail located at the tip of the drainage catheter.

In some embodiments, a system may come with the conduit assembled with the base (e.g., glued in place).

FIG. 6 depicts an embodiment of external fixation system 100 for percutaneous drainage procedures, wherein the external fixation system is coupled to drainage catheter 160 before the drainage catheter is positioned. Base 110 may be precoupled to the tube. In some embodiments, at least one suture is fixed between the screw and the tube. A second suture may be placed through a side hole in the base and exit through one of the side bars or spokes as depicted in FIG. 6. The bar may include small opening 240 (e.g., trap door), by pressing this segment, the suture will be fixed in placed keeping the loop of the pigtail formed. The cap in the back of the base may be attached to one of the bars using a line 250.

In some embodiments, an operator may select the length of the conduit based on the patient's existing urinary or biliary drain. Different lengths and diameters of drain tubes may be fabricated to meet the patient's needs.

In some embodiments, method of installing an external fixation system for percutaneous drainage procedures may include advancing a drainage tube over a guidewire under fluoroscopic guidance. The wire may be removed. The operator may secure the locking mechanism by making a knot with the sutures that will be fixed to the transverse bar in the back of the base. The sutures will be cut with scissors and the cap will be attached. Another way to lock the pigtail consists of a suture that is fixed to the base, a second suture exits through the side of the base and it is locked by pressing on a securing bar that holds the suture in place.

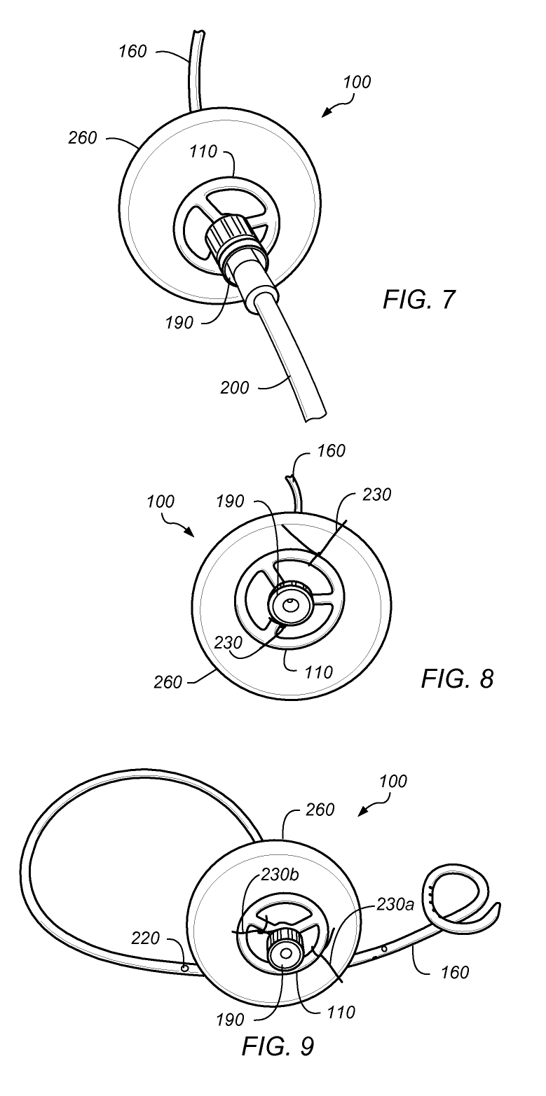

FIG. 7 depicts an embodiment of external fixation system 100 for percutaneous drainage procedures, wherein the external fixation system is coupled to a drainage catheter and “externally” to tissue 260 coupled to a drainage bag using the second coupler (e.g., a luer lock as depicted in FIG. 7). In some embodiments, the second coupler may include a coupling mechanism including external screw threads, internal screw threads, a female luer fitting, a male luer fitting, a push-to-connect fitting, or a spring-tension loaded trap-door fitting.

FIG. 8 depicts an embodiment of external fixation system 100 for percutaneous drainage procedures, wherein the external fixation system is coupled to tissue 260 using sutures. FIG. 9 depicts an embodiment of external fixation system 100 for percutaneous drainage procedures and a drainage catheter, wherein the external fixation system is coupled to tissue 260 using sutures. As is depicted the system may be coupled using at least two sutures. The sutures may couple the rim to a subject. First suture 230a may couple the rim such that the system is inhibited from moving in at least a first direction. Second suture 230b may couple the rim such that the system is inhibited from moving in at least a second direction. The first and second directions may be different directions. The first and second directions may include longitudinally and laterally.

The devices described herein address the unmet need to secure the position of an in-dwelling drainage tube on a patient's skin both laterally and longitudinally and in a manner consistent with easy drainage, maintenance and cleaning of the placed catheter, patient's confidence and comfort and safety. The devices described herein allow the subject to have the drainage tube in a secure position without a very visible and unnecessary excess portion of the drain protruding well beyond the skin surface. This system will also prevent drainage tube dislodgement and skin infection and will allow easy drainage of fluids and flushing (cleaning) of the drain tube/catheter.

In this patent, certain U.S. patents, U.S. patent applications, and other materials (e.g., articles) have been incorporated by reference. The text of such U.S. patents, U.S. patent applications, and other materials is, however, only incorporated by reference to the extent that no conflict exists between such text and the other statements and drawings set forth herein. In the event of such conflict, then any such conflicting text in such incorporated by reference U.S. patents, U.S. patent applications, and other materials is specifically not incorporated by reference in this patent.

Further modifications and alternative embodiments of various aspects of the invention will be apparent to those skilled in the art in view of this description. Accordingly, this description is to be construed as illustrative only and is for the purpose of teaching those skilled in the art the general manner of carrying out the invention. It is to be understood that the forms of the invention shown and described herein are to be taken as the presently preferred embodiments. Elements and materials may be substituted for those illustrated and described herein, parts and processes may be reversed, and certain features of the invention may be utilized independently, all as would be apparent to one skilled in the art after having the benefit of this description of the invention. Changes may be made in the elements described herein without departing from the spirit and scope of the invention as described in the following claims.

What is claimed is:

1. An external fixation system for percutaneous drainage procedures comprising:
a base, comprising:
a first side, wherein the first side is positioned adjacent an exterior surface of a subject during use;
a first coupler coupled to the first side of the base;
a second side opposite the first side; and
an opening extending through the first coupler and the base such that the first coupler is in fluid communication with the second side of the base;
wherein the first coupler is couplable to a conduit during use such that the conduit is in fluid communication with the second side of the base.

2. The system of claim 1, wherein the base comprises a metallic and/or a non-metallic material.

3. The system of claim 1, wherein at least a portion of the conduit extends into an interior of the subject.

4. The system of claim 1, wherein the conduit comprises a drain tube.

5. The system of claim 1, wherein the conduit comprises a catheter.

6. The system of claim 1, wherein the first coupler is fluidly connected to the conduit via screw threads.

7. The system of claim 1, wherein the first coupler is fluidly connected to the conduit via teeth.

8. The system of claim 1, wherein the base is coupled to the exterior surface of the subject using sutures.

9. The system of claim 1, wherein the base coupled to the exterior surface of the subject using a hydrocolloid layer configured to absorb excess moisture around a perimeter of the base.

10. The system of claim 1, wherein the base is coupled to the exterior surface of the subject using a double-sided hydrophilic adhesive layer configured to absorb excess moisture around the perimeter of the base.

11. The system of claim 1, wherein the base coupled to the exterior surface of the subject using a double-sided hydrophobic adhesive layer configured to repel excess moisture around the perimeter of the base.

12. The system of claim 1, wherein the second side of the base comprises a second coupler configured to facilitate the attachment of a drainage bag or a mechanism to flush or evacuate a drainage catheter.

13. The system of claim 12, wherein the second coupler comprises external screw threads.

14. The system of claim 12, wherein the second coupler comprises internal screw threads.

15. The system of claim 12, wherein the second coupler comprises a female luer fitting.

16. The system of claim 12, wherein the second coupler comprises a male luer fitting.

17. The system of claim 12, wherein the second coupler comprises a push-to-connect fitting.

18. The system of claim 12, wherein the second coupler comprises a spring-tension loaded trap-door fitting.

19. The system of claim 1, wherein the base further comprises a rim extending out and around at least a portion of the base.

20. The system of claim 1, wherein the base further comprises a rim extending out and around at least a portion of the base, wherein the rim is configured to inhibit the base from entering an interior of the subject.

21. The system of claim 1, wherein the base further comprises a rim extending out and around at least a portion of the base, and wherein the rim comprises one or more openings such that the base is coupled to the exterior surface of the subject.

22. The system of claim 1, wherein the base further comprises a rim extending out and around at least a portion of the base, and wherein the rim comprises one or more openings such that cleaning of the exterior surface of the subject is facilitated during use.

23. A method for positioning an external fixation system for percutaneous drainage procedures comprising:
positioning at least a portion of a conduit in a subject through an opening in the subject;
coupling a first coupler on a first side of a base of an external fixation system to a first end of the conduit such that at least a portion of the external fixation system is positioned externally of the subject;
inhibiting conveyance of the eternal fixation system through the opening using a rim coupled to the base, wherein the rim has a diameter greater than the opening; and
coupling the rim to an external surface of the subject such that rim is inhibited from moving in at least two different directions.

24. The method of claim 23, further comprising cleaning the external surface beneath and/or adjacent the rim using one or more openings extending through the rim.
